(19) United States
(12) Patent Application Publication
(10) Pub. No.: US 2022/0211539 A1
(43) Pub. Date: Jul. 7, 2022

(54) SCROTUM PROPHYLACTIC DEVICE WITH A FLAVOR STRIP

(71) Applicants: Melissa Mae Andrews, Reno, NV (US); Jeffrey Dean Parker, Othello, WA (US); Theresa Marie Hernandez, Sacramento, CA (US)

(72) Inventors: Melissa Mae Andrews, Reno, NV (US); Jeffrey Dean Parker, Othello, WA (US); Theresa Marie Hernandez, Sacramento, CA (US)

(21) Appl. No.: 17/141,878

(22) Filed: Jan. 5, 2021

(51) Int. Cl. A61F 6/04

(52) U.S. Cl. CPC A61F 6/04; A61H 19/32; A61F 2006/047

(57) ABSTRACT

A prophylactic device for protecting a scrotum of a human male. The device includes a sack configured to contain a scrotum of a human male, and made from low-density polyethylene (LDPE). The device also includes a flavor strip running up and down an outside surface of the sack. Optionally attached to an opening of the sack is a silicone ring that has at least one sleeve to receive and secure a vibrator. The silicone ring can be stretched so as to be worn around the upper portion of the scrotum, or a portion of a penis near the scrotum. The invention empowers both giver and receiver to partake in scrotal licking, without worrying about STD transmission.

### FIELD OF THE INVENTION

This invention relates generally to prophylactic devices for protecting a portion of a human male's genitalia, and specifically to prophylactic devices for providing a barrier against disease transmission to or from a human male's scrotum.

### BACKGROUND OF THE INVENTION

An important reason why many women feel uncomfortable with giving oral sex to a man—particularly where new sexual partners are concerned—is sexually transmitted disease. Today, many men are also concerned about exposing their partner to disease transmission due to intimate contact.

Many times when one engages in oral sex, one does not know the disease status of one's partner. Contrary to what many people believe, disease transmission during oral sex is a very real danger. For example, the oral transmission of herpes simplex, syphilis, gonorrhea, chlamydia, and a host of other lesser-known STDs is common.

### SUMMARY OF THE INVENTION

The invention is a scrotum prophylactic device that protects the scrotum from bodily fluids, such as saliva, and other secretions, such as vaginal secretions. The device includes a sack sized and shaped so as to contain a typical human male scrotum, the sack being made from low-density polyethylene (LDPE), optionally having an elastic band around the opening. Thus, when the device includes an elastic band, the device has an opening that functions like the opening of a shower cap.

The invention empowers giver and receiver alike to partake in scrotal licking, without worrying about STD transmission. Further, the scrotum prophylactic invention addresses this problem in a manner that does not stifle the enjoyment of scrotal licking and sucking for the recipient, while making the experience almost 100% safe and more enjoyable for provider and recipient alike. Thus, the invention allows the male recipient of oral stimulation to feel the probing and warmth of the oral stimulation provider's tongue, without the risk of direct tongue-to-genital contact.

The scrotum prophylactic of the invention allows and encourages a person to have more oral sex, licking, and/or sucking of the scrotum of a male, especially T-bagging, i.e., taking a man's testicles into one's mouth.

The scrotum prophylactic invention provides a prophylactic/erotic enhancement breakthrough—one that serves the needs and preferences of both women and men.

The flavor strip allows a woman to perform scrotal licking while enjoying the aroma and taste of the flavor strip, such as cherry, vanilla, chocolate, raspberry, or other generally pleasant flavor. Meanwhile, her male partner derives almost all of the pleasures of oral sex, without any worries about disease transmission to or from his scrotum.

The scrotum prophylactic of the invention is also useful for gay and bisexual men.

A general aspect of the invention is a prophylactic device for protecting a scrotum of a human male. The prophylactic device includes: a sack having an opening, the sack configured to substantially contain a scrotum of a human male, the sack being made from low-density polyethylene (LDPE), the opening including an elastic band that is configured to constrict around an upper portion of the scrotum; and a flavor strip running along an outside surface of the sack.

In some embodiments, the prophylactic device further includes: a silicone ring attached to the opening of the sack, the silicone ring configured to be stretched so as to be worn around one of: the upper portion of the scrotum; or a portion of a penis proximal to the scrotum.

In some embodiments, the silicone ring includes at least one sleeve, the sleeve configured to receive and secure a vibrator.

In some embodiments, the silicone ring includes two sleeves, each sleeve configured to receive and secure a vibrator, a first sleeve being on a top portion of the silicone ring, and a second sleeve being on a bottom portion of the silicone ring.

Another general aspect of the invention is a prophylactic device for protecting a scrotum of a human male, this prophylactic device including: a sack having an opening, the sack configured to substantially contain a scrotum of a human male, the sack being made from low-density polyethylene (LDPE), the opening including an elastic band that is configured to constrict around an upper portion of the scrotum; a flavor strip running along an outside surface of the sack; and a silicone ring attached to the opening of the sack, the silicone ring configured to be stretched so as to be worn around one of: the upper portion of the scrotum; or a portion of a penis proximal to the scrotum.

In some embodiments, the silicone ring includes at least one sleeve, the sleeve configured to receive and secure a vibrator.

In some embodiments, the silicone ring includes two sleeves, each sleeve configured to receive and secure a vibrator, a first sleeve being on a top portion of the silicone ring, and a second sleeve being on a bottom portion of the silicone ring.

Yet another general aspect of the invention is a prophylactic device for protecting a scrotum of a human male, this prophylactic device including: a sack having an opening, the sack configured to substantially contain a scrotum of a human male, the sack being made from low-density polyethylene (LDPE), the opening including an elastic band that is configured to constrict around an upper portion of the scrotum; a flavor strip running along an outside surface of the sack; and a silicone ring including at least one sleeve configured to receive and secure a vibrator, the silicon ring attached to the opening of the sack, the silicone ring configured to be stretched so as to be worn around one of: the upper portion of the scrotum; or a portion of a penis proximal to the scrotum.

In some embodiments, the silicone ring includes two sleeves, each sleeve configured to receive and secure a vibrator, a first sleeve of the two sleeves being on a top portion of the silicone ring, and a second sleeve of the two sleeves being on a bottom portion of the silicone ring.

### BRIEF DESCRIPTION OF THE DRAWINGS

The invention will be more fully understood from the following Detailed Description, in conjunction with the following figures, wherein:

FIG. 1 is a front view of the prophylactic device for protecting a scrotum of a human male.

FIG. 2 is a front view of the prophylactic device of FIG. 1, enclosing a scrotum of a human male.

FIG. 3 is a front view of the prophylactic device and the scrotum of FIG. 1, the prophylactic device also including a flavor strip.

FIG. 4 is a side view of the prophylactic device with the flavor strip of FIG. 1, the prophylactic device also including a silicone ring with two sleeves for supporting two respective bullet vibrators, the silicone ring attached to a point along the opening of the prophylactic device so as to be worn around a portion of a penis near the scrotum.

FIG. 5 is a front view of a prophylactic device enclosing a scrotum of a human male, also including a silicone ring with two sleeves for supporting two respective bullet vibrators, the silicone ring attached all along the opening of the prophylactic device so as to be worn around the upper portion of the scrotum.

FIG. 6 is a front view of the prophylactic device of FIG. 5, the silicone ring having a bullet vibrator inserted in each of the two sleeves of the ring, the silicone ring attached all along the opening of the prophylactic device so as to be worn around the upper portion of the scrotum.

FIG. 7 is a side view of the prophylactic device with the flavor strip of FIG. 4, the silicone ring supporting two respective bullet vibrators.

FIG. 8 is a side view of the prophylactic device with the flavor strip of FIG. 4, showing how the ring is worn around a portion of a penis near the scrotum.

FIG. 9 is a side view of the prophylactic device with the flavor strip of FIG. 8, showing the silicone ring supporting two respective bullet vibrators, and showing how the ring is worn around a portion of a penis near the scrotum.

FIG. 10 is side view of an embodiment of a prophylactic device with the flavor strip, showing a silicone ring without any sleeves or bullet vibrators, and showing how the ring without sleeves is worn around a portion of a penis near the scrotum.

### DETAILED DESCRIPTION

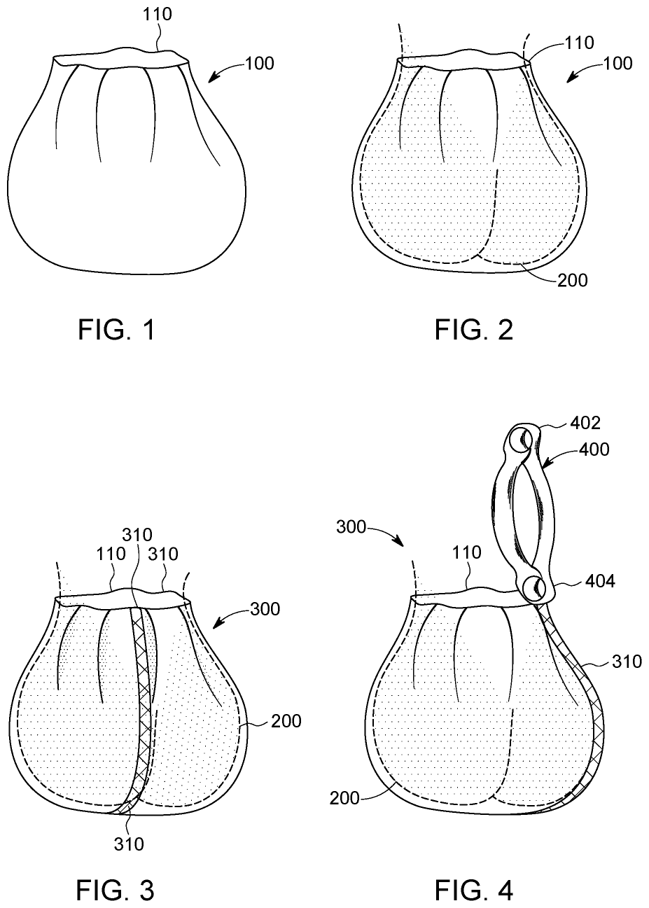

With reference to FIG. 1, a prophylactic device 100 is shown for protecting a scrotum of a human male. The prophylactic device 100 provides a sanitary barrier to permit licking sensations to reach the scrotum, without allowing potential pathogens to pass from the scrotum to the person licking, and without allowing potential pathogens to pass from the person licking to the scrotum. The prophylactic device 100 is made from low-density polyethylene (LDPE). LDPE is commonly used to make plastic bags. The prophylactic device 100 has an opening 110 at the top that is configured to expand resiliently so as to permit a male scrotum to be inserted into the prophylactic device 100, the opening 110 then returning to its original size. An optional elastic band (not shown, but similar to the elastic band on a typical shower cap) can be secured to the device 100 around the opening 110 to add elasticity.

Referring to FIG. 2, the prophylactic device 100 is shaped and sized so as to contain and closely conform to the contours of a typical male scrotum 200.

FIG. 3 is a front view of a prophylactic device 300 and the scrotum 200 of FIG. 2, the prophylactic device 300 also including a flavor strip 310 that extends over the front side, bottom, and back side of the prophylactic device 300, from a front portion of the opening 110 to a rear portion of the opening 110.

FIG. 4 is a side view of the prophylactic device 300 with the flavor strip 310, the prophylactic device 300 also including a silicone ring 400 with two sleeves 402, 404 for supporting two respective bullet vibrators (not shown), the silicone ring 400 attached to a point along the opening 110 of the prophylactic device 300 so as to be worn around a portion of a penis (not shown) near the scrotum 200.

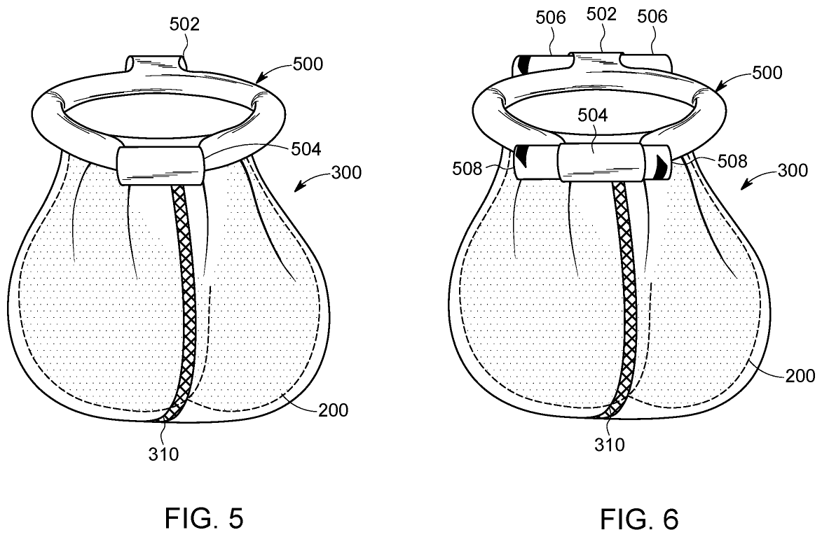

FIG. 5 is a front view of a prophylactic device 300 enclosing a scrotum 200 of a human male, also including a silicone ring 500 with two sleeves 502, 504 for supporting two respective bullet vibrators (not shown), the silicone ring 500 attached all along the opening 110 (not shown) of the prophylactic device 300 so as to be worn around the upper portion of the scrotum 200 under the penis (not shown). The flavor strip 310 extends downward, and upward towards the ring 500.

FIG. 6 is a front view the prophylactic device 300 of FIG. 5, the silicone ring 500 having a bullet vibrator 506, 508 inserted in each of the two sleeves 502, 504 of the ring 500. The silicone ring 500 is attached all along the opening 110 (not shown in this FIG) of the prophylactic device 300 so as to be worn around the upper portion of the scrotum 200.

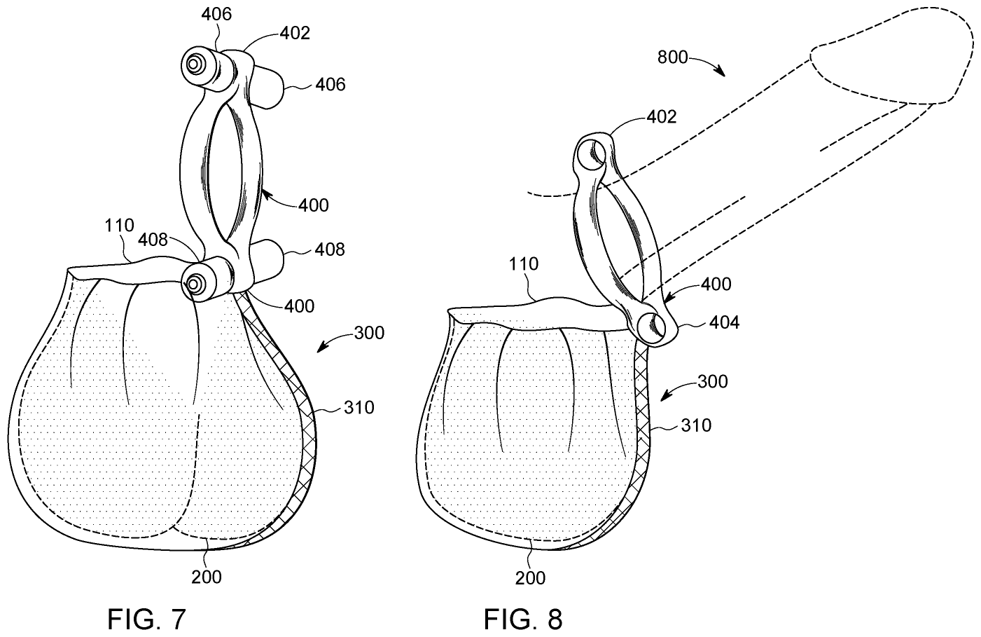

FIG. 7 is a side view of the prophylactic device 300 with the flavor strip 310 of FIG. 4. The two sleeves 402, 404 of the silicone ring 400 support the two respective bullet vibrators 406, 408. The ring 400 is worn around the man's penis (not shown in this FIG.)

FIG. 8 is a side view of the prophylactic device 300 with the flavor strip 310 of FIG. 4, also showing how the ring 400 is worn around a portion of a penis 800 near the scrotum 200. The ring 400 is attached to a front portion of the opening 110 of the device 300. The flavor strip 310 is shown. No vibrating bullets are inserted in the sleeves 402, 404 in this view. The ring 400 is stretchy, and can be used with or without the vibrating bullets.

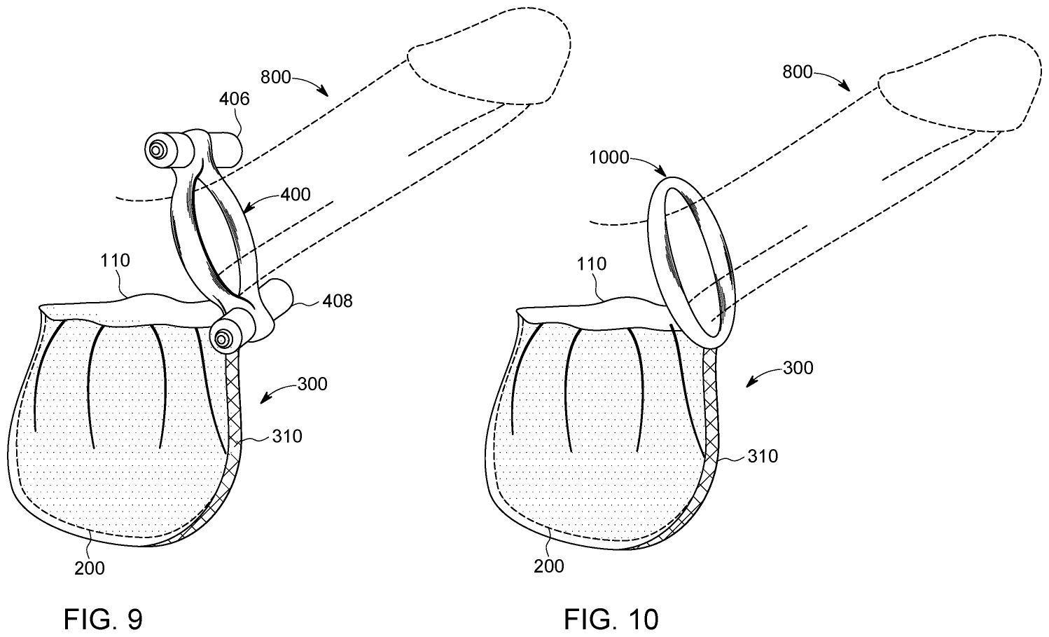

FIG. 9 is a side view of the prophylactic device 300 with the flavor strip 310 of FIG. 8, showing the silicone ring 400 supporting two respective bullet vibrators 406 and 408. Also shown is how the ring 400 is worn around a portion of a penis 800 near the scrotum 200.

FIG. 10 is side view of an embodiment 300 of a prophylactic device with the flavor strip 310, showing a silicone ring 1000 without any sleeves or bullet vibrators, and showing how the ring 1000 without sleeves is worn around a portion of a penis 800 near the scrotum 200. This ring 1000 is attached to a forward portion of the opening 110 of the device 300.

Other modifications and implementations will occur to those skilled in the art without departing from the spirit and the scope of the invention as claimed. Accordingly, the above description is not intended to limit the invention, except as indicated in the following claims.

What is claimed is:

1. A prophylactic device for protecting a scrotum of a human male, the prophylactic device comprising:
a sack having an opening, the sack configured to substantially contain a scrotum of a human male, the sack being made from low-density polyethylene (LDPE), the opening including an elastic band that is configured to constrict around an upper portion of the scrotum; and
a flavor strip running along an outside surface of the sack.

2. The prophylactic device of claim 1, further including:
a silicone ring attached to the opening of the sack, the silicone ring configured to be stretched so as to be worn around one of:
the upper portion of the scrotum; or
a portion of a penis proximal to the scrotum.

3. The prophylactic device of claim 2, wherein the silicone ring includes at least one sleeve, the sleeve configured to receive and secure a vibrator.

4. The prophylactic device of claim 2, wherein the silicone ring includes two sleeves, each sleeve configured to receive and secure a vibrator, a first sleeve being on a top portion of the silicone ring, and a second sleeve being on a bottom portion of the silicone ring.

5. A prophylactic device for protecting a scrotum of a human male, the prophylactic device comprising:
a sack having an opening, the sack configured to substantially contain a scrotum of a human male, the sack being made from low-density polyethylene (LDPE), the opening including an elastic band that is configured to constrict around an upper portion of the scrotum;
a flavor strip running along an outside surface of the sack; and
a silicone ring attached to the opening of the sack, the silicone ring configured to be stretched so as to be worn around one of:
the upper portion of the scrotum; or
a portion of a penis proximal to the scrotum.

6. The prophylactic device of claim 5, wherein the silicone ring includes at least one sleeve, the sleeve configured to receive and secure a vibrator.

7. The prophylactic device of claim 5, wherein the silicone ring includes two sleeves, each sleeve configured to receive and secure a vibrator, a first sleeve being on a top portion of the silicone ring, and a second sleeve being on a bottom portion of the silicone ring.

8. A prophylactic device for protecting a scrotum of a human male, the prophylactic device comprising:
a sack having an opening, the sack configured to substantially contain a scrotum of a human male, the sack being made from low-density polyethylene (LDPE), the opening including an elastic band that is configured to constrict around an upper portion of the scrotum;
a flavor strip running along an outside surface of the sack; and
a silicone ring including at least one sleeve configured to receive and secure a vibrator, the silicon ring attached to the opening of the sack, the silicone ring configured to be stretched so as to be worn around one of:
the upper portion of the scrotum; or
a portion of a penis proximal to the scrotum.

9. The prophylactic device of claim 8, wherein the silicone ring includes two sleeves, each sleeve configured to receive and secure a vibrator,
a first sleeve of the two sleeves being on a top portion of the silicone ring, and
a second sleeve of the two sleeves being on a bottom portion of the silicone ring.
